(19) United States
(12) Patent Application Publication
(10) Pub. No.: US 2026/0207648 A1
(43) Pub. Date: Jul. 23, 2026

(54) NOVEL SMALL MOLECULE KRAS INHIBITOR: DRUGGING UNDRUGGABLE TARGETS FOR CANCER THERAPEUTICS

(71) Applicant: PILLAI UNIVERSAL LLC, Carson City, NV (US)

(72) Inventors: Baskaran PILLAI, Pittsburgh, PA (US); Dinesh M.G., Ambattur, Chennai (IN)

(21) Appl. No.: 19/139,081

(22) PCT Filed: Dec. 11, 2023
(86) PCT No.: PCT/IB2023/062486

§ 371 Date: Jun. 13, 2025

(30) Foreign Application Priority Data
Dec. 15, 2022 (IN) 202241072448

(51) Int. Cl. A61K 31/708; C07H 19/207

(52) U.S. Cl. CPC A61K 31/708; C07H 19/207

(57) ABSTRACT

The present invention discloses a anticancer activity exhibiting compound of structural formula I:

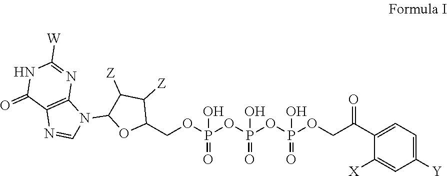

or a pharmaceutically acceptable salt thereof; in which
W is selected from a group comprising of substituted saturated/unsaturated branched/linear aliphatic amine, substituted aromatic amine, unsubstituted saturated/unsaturated branched/linear aliphatic amine, unsubstituted aromatic amine;
X is selected from a group comprising of H, substituted/unsubstituted saturated/unsaturated branched/linear aliphatic alkyl group with at least one carbon atom, substituted/unsubstituted saturated/unsaturated branched/linear aliphatic alkoxy group with at least one carbon atom, substituted/unsubstituted aryl group, substituted/unsubstituted aryloxy group;
Y is selected from a group comprising of H, substituted/unsubstituted saturated/unsaturated branched/linear aliphatic alkyl group bonded to a carbonyl group, substituted/unsubstituted aryl group bonded to a carbonyl group;
Z is selected from a group comprising of H, OH.

### FIELD OF THE INVENTION

The present invention relates to novel molecule exhibiting anti-cancer activity. More particularly the present invention relates to a novel small molecule capable of inhibition of Ras downstream signaling and cellular proliferation in cancer cells expressing wild type and K-Ras G12C, G12D and G12S, and N-Ras Q61K mutants.

### BACKGROUND OF THE INVENTION

RAS genes encode small GTPases essential for proliferation, differentiation, and survival of mammalian cells. RAS gene mutations are associated with approximately 30% of all human cancers. However, based on measurements reported three decades ago of Ras protein affinities to GTP in the 10-20 picomolar range, it has been accepted in the scientific and medical communities that Ras proteins are undruggable targets.

Ras proteins are encoded by three ubiquitously expressed genes (H-RAS, K-RAS, and N-RAS)(Parker and Mattos, 2018). They couple cell surface receptors to intracellular effector pathways and are key regulators of cellular growth and differentiation (Simanshu et al., 2017; Stephen et al., 2014). The binding of GTP and GDP, respectively, cycle Ras proteins between ‘on’ and ‘off’ signaling conformations. Under physiological conditions, the transition between these two states has been shown to be regulated by guanine nucleotide exchange factors (GEFs), which promote the activation of Ras proteins by stimulating the release of bound GDP and binding of GTP, and by GTPase-activating proteins (GAPs), which accelerate Ras-mediated GTP hydrolysis (Simanshu et al., 2017; Milburn et al., 1990; Shieh, 2019; Hunter et al., 2015). Ras proteins are a subset of the Ras GTPase superfamily which consists of over 150 human members which exhibit significant sequence homology in their GTP binding site, including Rac-1, Rho-A and cdc42 (Wennerberg et al., 2005; Cox and Der, 2010). Approximately 30% of all human cancers are associated with mutations in Ras proteins (Prior et al., 2012; Hobbs et al., 2016; Pylayeva-Gupta et al., 2011). K-Ras is the most mutated Ras isoform. K-Ras mutations are frequently detected in pancreatic, colorectal and lung tumors (Prior et al., 2012; Hobbs et al., 2016; Cox et al., 2014). The most frequent K-Ras mutations are in residues Gly12, Gly13 and Gln61 (Hobbs et al., 2016; Li et al., 2018). N-Ras is mutated in about 20% of all melanoma patients (Jenkins and Sullivan, 2016). H-Ras mutations are comparatively rare (Prior et al., 2012). A serious unmet medical need persists for patients with diseases, particularly cancer, associated with mutated RAS genes. A number of small molecules specifically targeting the K-Ras G12C mutant via a covalent mechanism have been identified, two of which are currently in clinical development (McCormick, 2020; Grapsa and Syrigos, 2020). However, potent small molecules targeting other mutants or wild-type Ras proteins have not been published. This is likely in major part due to the generally accepted paradigm that small molecule drugs cannot be developed which can compete with guanine nucleotides for binding to the GTP/GDP binding site of Ras, a conception based on studies from the 1990's reporting GTP binding affinities for Ras proteins in the 10-20 pM range (John et al., 1990; John et al., 1993). Therefore an attempt has been made to develop a small molecule drug capable of inhibition of Ras downstream signaling and cellular proliferation in cancer cells expressing wild type and K-Ras G12C, G12D and G12S, and N-Ras Q61K mutants.

### OBJECT OF THE INVENTION

The main object of the present invention relates to novel small molecule exhibiting anti cancer activity.

Another object of the present invention is to synthesize novel small molecule exhibiting anti cancer activity.

Yet another object of the present invention is to synthesize novel small molecule capable of inhibition of Ras downstream signaling and cellular proliferation in human pancreatic and non-small cell lung cancer cells expressing wild type and K-Ras G12C, G12D and G12S, and N-Ras Q6JK mutants.

Further object of the present invention is to utilize the developed small molecule for subjects suffering from carcinoma.

### BRIEF DESCRIPTION OF DRAWINGS

FIG. 1 depicts the cell proliferation assay—MTT

FIG. 2 depicts the effect of COMPOUND I on inducing Apoptosis—Immunoblot assay

FIG. 3 depicts the effect of COMPOUND I on inducing Cell Cycle arrest

FIG. 4 depicts that effect of COMPOUND I in increasing cell cyclins inhibitor

FIG. 5 depicts the effect of COMPOUND I on cell cyclin checkpoints

FIG. 6 depicts the effect of COMPOUND I on mTor pathway

FIG. 7 depicts the effect of COMPOUND I on KRAS Mutated cell lines

FIG. 8 depicts the effect of COMPOUND I on Ras downstream Signalling

FIG. 9 depicts the effect of COMPOUND I on inhibition of the Ras-GTP complex

FIG. 10 depicts the JH NMR spectra of the compound I of the present invention

FIG. 11 depicts the 13CNMR Spectra of the compound I of the present invention

FIG. 12 depicts the 2D COSY Spectrum of the compound I of the present invention

FIG. 13 depicts the IR Spectrum of the compound I of the present invention

### SUMMARY OF THE INVENTION

The present invention discloses a anticancer activity exhibiting compound of structural formula I:

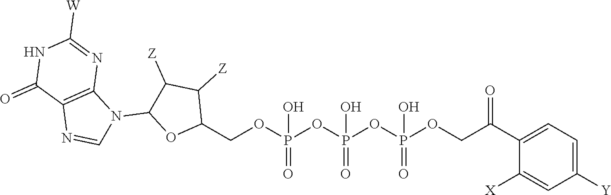

Formula I
or a pharmaceutically acceptable salt thereof; in which
   W is selected from a group comprising of substituted saturated/unsaturated branched/linear aliphatic amine, substituted aromatic amine, unsubstituted saturated/unsaturated branched/linear aliphatic amine, unsubstituted aromatic amine;
   X is selected from a group comprising of H, substituted/unsubstituted saturated/unsaturated branched/linear aliphatic alkyl group with at least one carbon atom, substituted/unsubstituted saturated/unsaturated branched/linear aliphatic alkoxy group with at least one carbon atom, substituted/unsubstituted aryl group, substituted/unsubstituted aryloxy group;
   Y is selected from a group comprising of H, substituted/unsubstituted saturated/unsaturated branched/linear aliphatic alkyl group bonded to a carbonyl group, substituted/unsubstituted aryl group bonded to a carbonyl group;
   Z is selected from a group comprising of H, OH.

### DETAILED DESCRIPTION OF THE INVENTION

### Definitions

Alkyl” means saturated carbon chains which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. In one embodiment of the present invention, alkyl is methyl.

“Aryl” means a monocyclic, bicyclic or tricyclic carbocyclic aromatic ring or ring system containing 5-14 carbon atoms, wherein at least one of the rings is aromatic. Examples of aryl include phenyl and naphthyl. In one embodiment of the present invention, aryl is phenyl.

“Alkoxy” refers to a group of the formula —ORₐ where Rₐ is an alkyl, alkenyl or alkynyl as defined above containing one to twelve carbon atoms. Unless stated otherwise specifically in the specification, an alkoxy group can be optionally substituted.

“Aryloxy” refers to a group of the formula —OAr where Ar is an monocyclic, bicyclic or tricyclic carbocyclic aromatic ring or ring system containing 5-14 carbon atoms, wherein at least one of the rings is aromatic. Examples of aryl include phenyl and naphthyl. In one embodiment of the present invention, aryl is phenyl.

In choosing compounds of the present invention, one of ordinary skill in the art will recognize that the various substituents, are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

It will be understood that, as used herein, references to the compounds of the present invention are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, 0-pivaloyl, O-benzoyl, and O-aminoacyl, can be employed. Included are those esters and acyl groups known in the art for modifying the solubility or hydrolysis characteristics for use as sustained-release or prodrug formulations.

The term “therapeutically effective” applied to dose or amount refers to that quantity of a compound or pharmaceutical formulation that is sufficient to result in a desired clinical benefit after administration to a patient in need thereof.

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally the present invention discloses novel therapeutic compounds exhibiting anticancer activity.

In one of the preferred embodiment, the present invention shall discloses a compound of structural formula I:

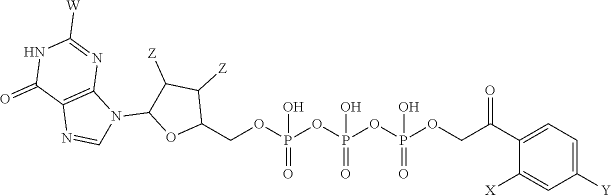

Formula I
or a pharmaceutically acceptable salt thereof; in which
   W is selected from a group comprising of substituted saturated/unsaturated branched/linear aliphatic amine, substituted aromatic amine, unsubstituted saturated/unsaturated branched/linear aliphatic amine, unsubstituted aromatic amine;
   X is selected from a group comprising of H, substituted/unsubstituted saturated/unsaturated branched/linear aliphatic alkyl group with at least one carbon atom, substituted/unsubstituted saturated/unsaturated branched/linear aliphatic alkoxy group with at least one carbon atom, substituted/unsubstituted aryl group, substituted/unsubstituted aryloxy group;
   Y is selected from a group comprising of H, substituted/unsubstituted saturated/unsaturated branched/linear aliphatic alkyl group bonded to a carbonyl group, substituted/unsubstituted aryl group bonded to a carbonyl group;
   Z is selected from a group comprising of H, OH.

According to the invention, in the compound of formula 1 the W is substituted aromatic amine or a pharmaceutically acceptable salt thereof.

As per the invention, in the compound of formula 1 the W is N-bonded p-Hydroxyaniline.

In accordance with the invention, in the compound of formula 1 the X is unsubstituted saturated linear aliphatic alkoxy group with at least one carbon atom or a pharmaceutically acceptable salt thereof.

According to the invention, in the compound of formula 1 the X is Methoxy group. As per the invention, in the compound of formula 1 the Y is unsubstituted saturated linear aliphatic alkyl group bonded to a carbonyl group or a pharmaceutically acceptable salt thereof.

In accordance with the invention, in the compound of formula 1 the Y is Acetyl group.

According to the invention, in the compound of formula 1 the Z is hydroxyl group or a pharmaceutically acceptable salt thereof.

As per the invention, in the compound of formula 1 the Z is hydroxyl group.

In another preferred embodiment, the present invention shall disclose a comprises of structure of compound I

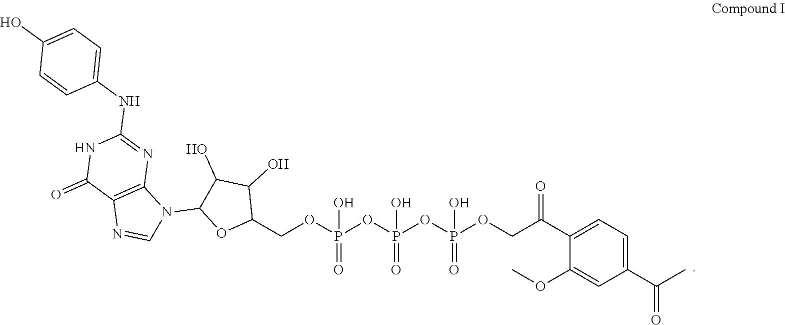 [SMILES: COc1cc(C(C)=O)ccc1C(=O)COP(=O)(O)OP(=O)(O)OP(=O)(O)OCC1OC(n2cnc3c(=O)[nH]c(Nc4ccc(O)cc4)nc32)C(O)C1O]

In yet another preferred embodiment, the present invention shall disclose a pharmaceutical composition comprising a therapeutically effective amount of compound of formula 1, or a therapeutically effective amount of pharmaceutically acceptable salt thereof, and a pharmaceutically acceptable carrier.

In further preferred embodiment, the present invention shall disclose a pharmaceutical composition comprising a therapeutically effective amount of compound I, and a pharmaceutically acceptable carrier.

The chemical structures for any of Formulae 1 can be prepared by routine chemistry based on the example structures provided herein.

In one embodiment, example compound 1 of Formula 1 can be prepared by the manufacturing process provided below.

### 1.1 Stage-1: Preparation of ABCD001

### Brief Manufacturing Process:

(5-(2-amino-6-oxo-1H-purin-9(6H)-yl)-3,4-dihydroxytetrahydrofuran-2-yl)methyl tetrahydrogen triphosphate reacts with BOC-anhydride in presence of base, THF and water to affords ABCD001.

### Route of Synthesis:

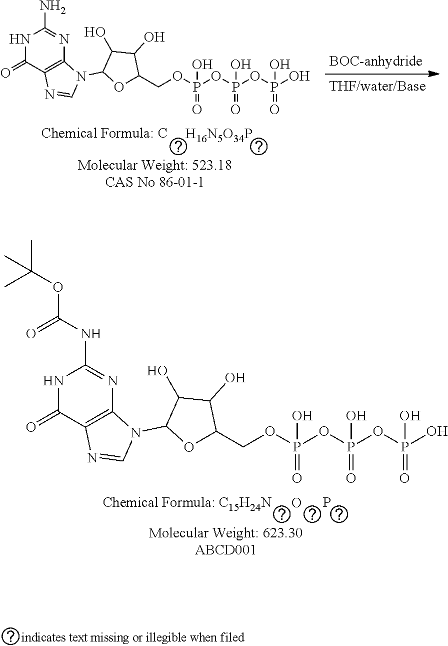

### 1.2 Stage-2: Preparation of ABCD002

### Brief Manufacturing Process:

ABCD001 reacts with 2-(4-acetyl-2-methoxyphenyl)-2-oxoethyl 4-methylbenzenesulfonatein presence of tetrabutylammoniumbromide and acetonitrile to afford ABCD002.

### Route of Synthesis:

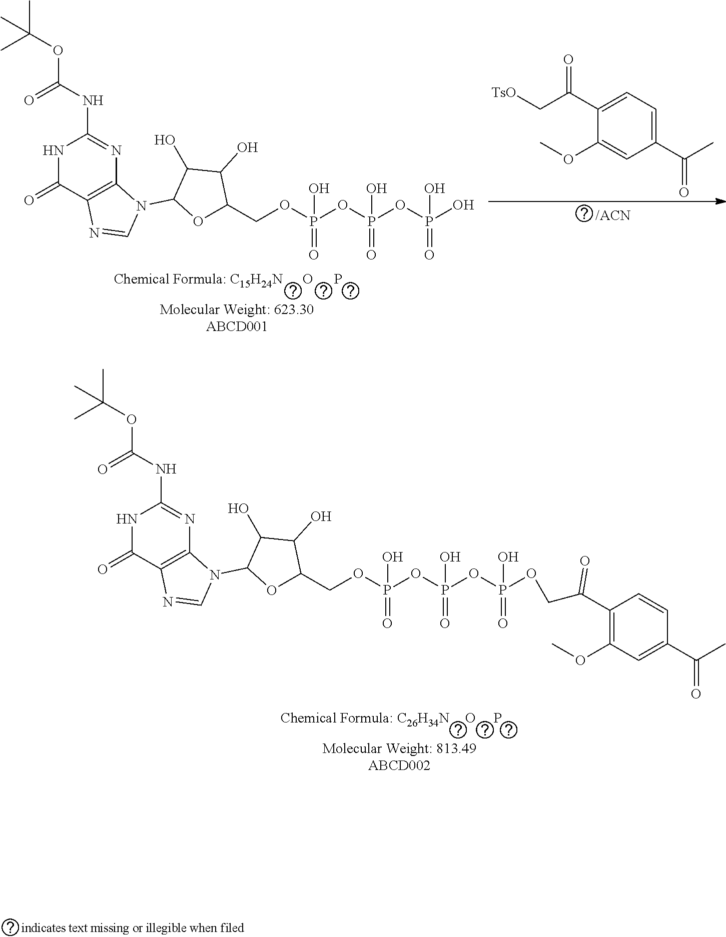

### 1.3 Stage-3: Preparation of ABCD003

### Brief Manufacturing Process:

ABCD002 reacts with hydrochloric acid in Dioxane to afford ABCD003.

### Route of Synthesis:

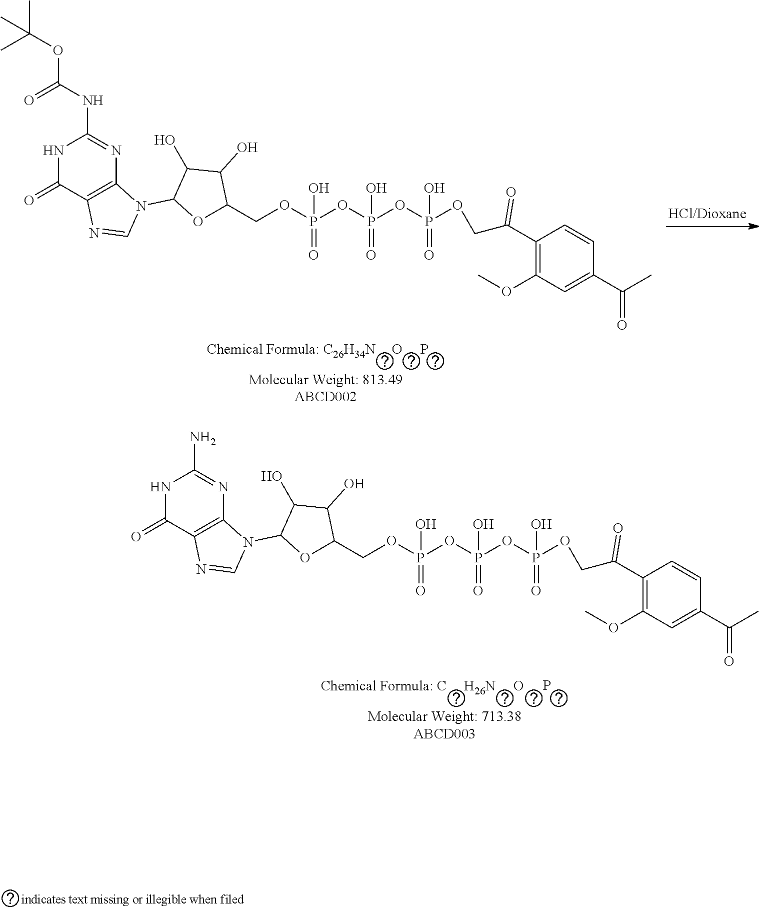

### 1.4 Stage-4: Preparation of ABCD004/Compound I (Target Molecule)

### Brief Manufacturing Process:

ABCD003 reacts with 4-bromo phenol in presence of copper catalyst, potassium carbonate and methanol to afford ABCD004/Compound I.

### Route of Synthesis:

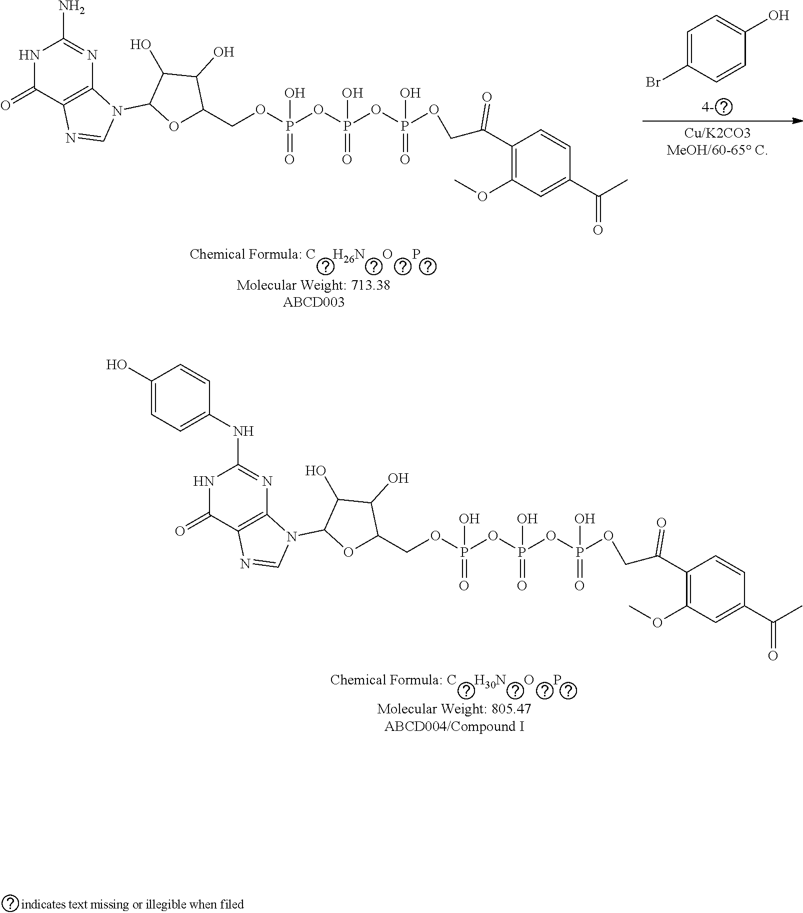

The synthesized compound I is next subject to molecular characterization to ascertain the structure of the compounds.

### Molecules Characterization:

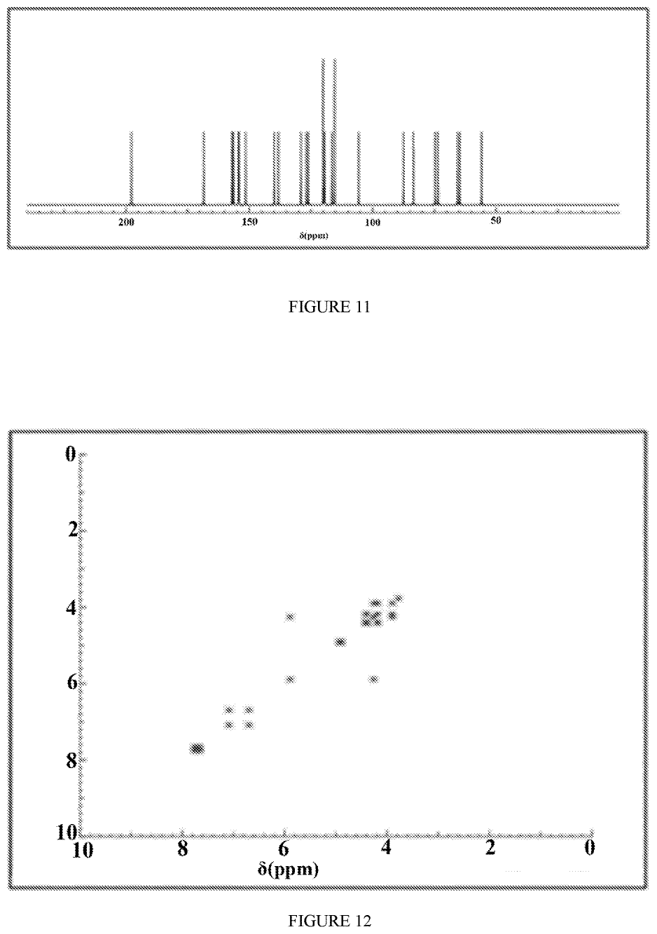

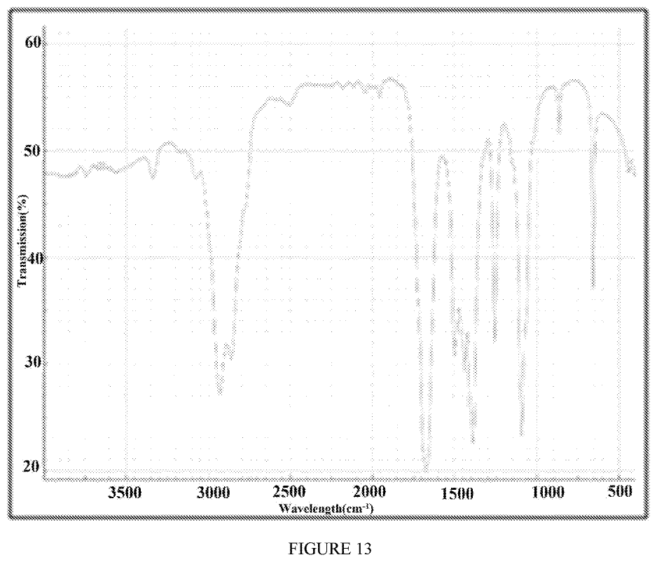

The purity of synthesized Compound I was verified by the melting point, Thin Layer Chromatography, HPLC, JR, and NMR analysis. From FIG. 10-13, the structure of the synthesized compound I is elucidated as follows.

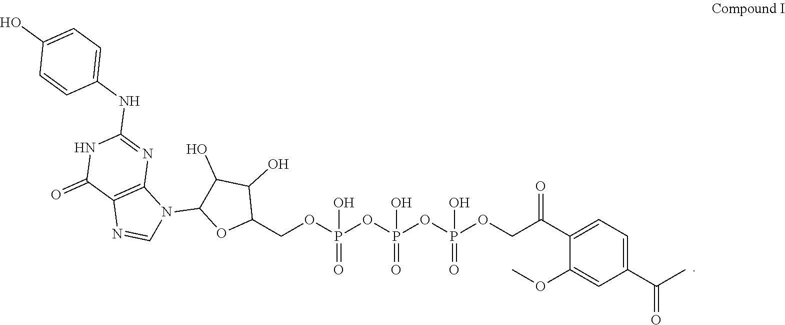 [SMILES: COc1cc(C(C)=O)ccc1C(=O)COP(=O)(O)OP(=O)(O)OP(=O)(O)OCC1OC(n2cnc3c(=O)[nH]c(Nc4ccc(O)cc4)nc32)C(O)C1O]

In some embodiments, the compound I may encompass both the cis- and trans-isomers. In some embodiments, the compound I may be a mixture of cis- and trans-isomers. In some embodiments, the compound I may be cis-isomer. In some embodiments, the compound I may be trans-isomer.

In some embodiments, the compound I may encompass either R or S stereoisomers and a mixture of stereoisomers. In some embodiments, the compound I may encompass both racemic isomers and enantiomeric isomers The compound I of the present inventions can be used to perform or provide any of the biological functions, described herein.

### Pharmaceutical Compositions

The present disclosure also includes pharmaceutical compositions comprising a therapeutically effective amount of compound I disclosed herein. In some embodiments, pharmaceutical compositions comprise a therapeutically effective amount of compound I or pharmaceutically acceptable salts thereof.

In various aspects, the amount of compound I, or a pharmaceutically acceptable salt thereof, can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be once administered daily or more frequently depending upon a number of factors, including the overall health of a patient, and the formulation and route of administration of the selected compound(s).

The compounds or pharmaceutical compositions of the present disclosure may be manufactured and/or administered in single or multiple unit dose forms.

In some embodiments, the compound I of the present disclosure are administered to a patient with a cancer and its related complications.

In certain embodiments, the compounds, and compositions described herein are administered in combination with one or more of anti cancer drug. Compound I of the present invention may be used in combination with other drugs that may also be useful in the treatment or amelioration of the diseases or conditions for which compounds of the present invention are useful. Such other drugs may be administered, by a route and in an amount commonly used therefor, contemporaneously or sequentially with a compound of the present invention.

When compound I of the present invention is used contemporaneously with one or more other drugs, a pharmaceutical composition in unit dosage form containing such other drugs and the compound of the present invention is preferred. However, the combination therapy also includes therapies in which the compound I of the present invention and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of the present invention.

Examples of other active ingredients that may be administered separately or in the same pharmaceutical composition in combination with compound I described herein include, but are not limited to altretamine, bendamustine, busulfan, carboplatin, chlorambucil, cisplatin, cyclophosphamide, dacarbazine, ifosfamide, mechlorethamine, melphalan, oxaliplatin, temozolomide, thiotepa and trabectedin

The present invention also provides a method for the treatment of cancer, which method comprises administration to a patient therapeutically effective amount Compound I of the present invention and an amount of one or more active ingredients, such that together they give effective relief.

In a further aspect of the present invention, there is provided a pharmaceutical composition comprising a Compound I of the present invention together with at least one pharmaceutically acceptable carrier or excipient.

Thus, according to a further aspect of the present invention there is provided the use of Compound I of the present invention for the manufacture of a medicament for the treatment of cancer. In a further or alternative aspect of the present invention, there is therefore provided a product comprising a Compound I of the present invention and one or more active ingredients as a combined preparation for simultaneous, separate or sequential use in the treatment of cancer.

It will be appreciated that for the treatment of cancer, a compound of the present invention may be used in conjunction with another pharmaceutical agent effective to treat that disorder.

The present invention also provides a method for the treatment of cancer, which method comprises administration to a patient in need of such treatment an amount of a compound I of the present invention and an amount of another pharmaceutical agent effective to threat that disorder, such that together they give effective relief.

### BIOLOGICAL EXAMPLES

### Example 1

### Cell Viability on Human Adenocarcinoma Cell Lines

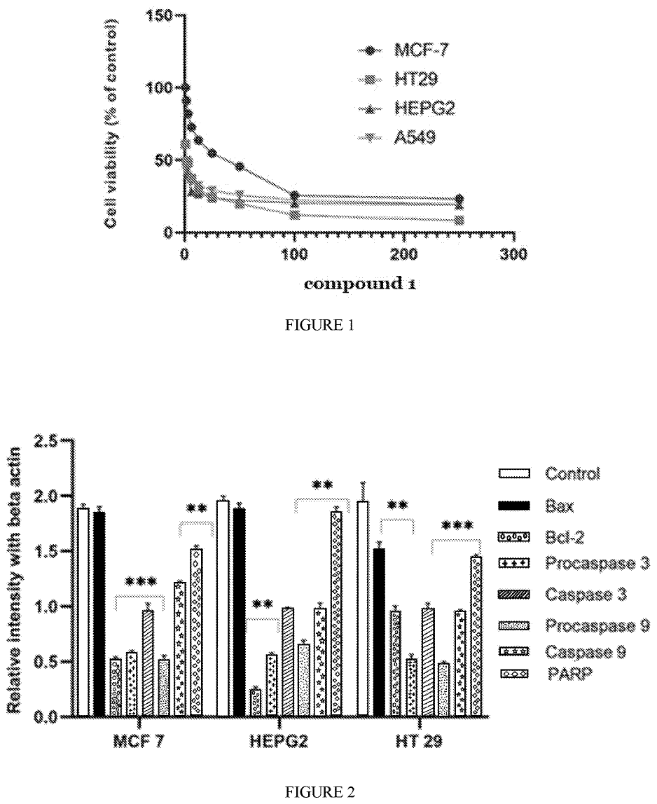

Several cell lines including human breast carcinoma (MCF7), colon adenocarcinoma (HT-29), Liver adenocarcinoma (HEP G2), Lung adenocarcinoma (A549) were treated with a range of COMPOUND I for 48 hours and the percentage of cell viability was determined by MTT assay. The average total viable cells were precisely calculated and anti-proliferative effects were determined. COMPOUND I inhibited the proliferation of all the cell lines in a dose-dependent manner (FIG. 1). A dose-dependent growth inhibition was observed at concentrations ranging from 250 to 0.97 nmol/L and each cell line had a different sensitivity. The drug were also tested on normal monkey Kidney epithelial cells. Expectedly normal Monkey Kidney epithelial cells were less sensitive to induced cytotoxicity.

### Example 2

### Effect of COMPOUND I Triggers Apoptotic Pathway

To confirm that COMPOUND I induced cell apoptosis, DNA fragmentation assay, was performed. Exposure of cells to COMPOUND I for 48 hours lead to DNA fragmentation as indicated by the typical ladder pattern of DNA in the agarose gels at all concentrations tested, whereas control showed no DNA fragmentation. Quantitative analysis revealed a significant increase of apoptosis in cells treated with COMPOUND I and higher doses of the drugs were found to exhibit pronounce cytotoxic and anti-proliferation effect. Furthermore, the expression levels of proteins related to apoptosis and cell cycle progression by western blotting were examined. Cells were treated with COMPOUND I at different concentrations and total proteins were isolated. Beta-Actin was used as an internal control. COMPOUND I supplementation down regulated expression of Bcl-2 a potent suppressor of apoptosis and induced expression of proapoptotic proteins Bax, caspase-9 and caspase-3 in a dose dependent manner (FIG. 2). The effect of drugs on the cleavage of procaspase 9 and procaspase 3 were examined. Western blot analysis demonstrated that cleavages of procaspase 9 and procaspase 3 were induced in a dose-dependent manner (FIG. 2). Caspase-3 is a key regulator of mitochondrial dependent and independent apoptotic pathways and activation of caspase-3 leads to cleavage of several substrates including PARP. Subsequently COMPOUND I induced the cleavage of PARP into 116 kDa and 84 kDa fragment as determined by western blot analysis.

### Example 3

### Effect of COMPOUND I on Cell Cycle Arrest

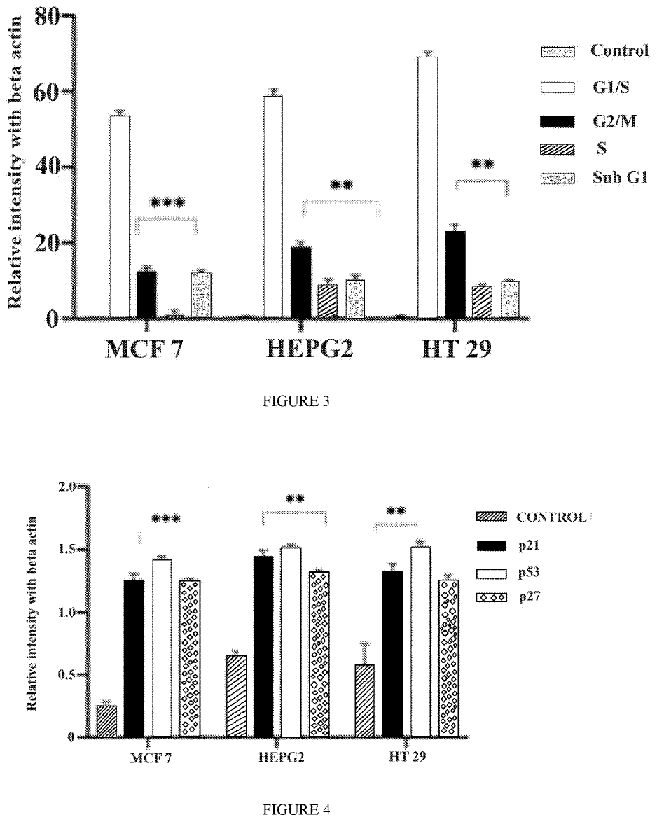

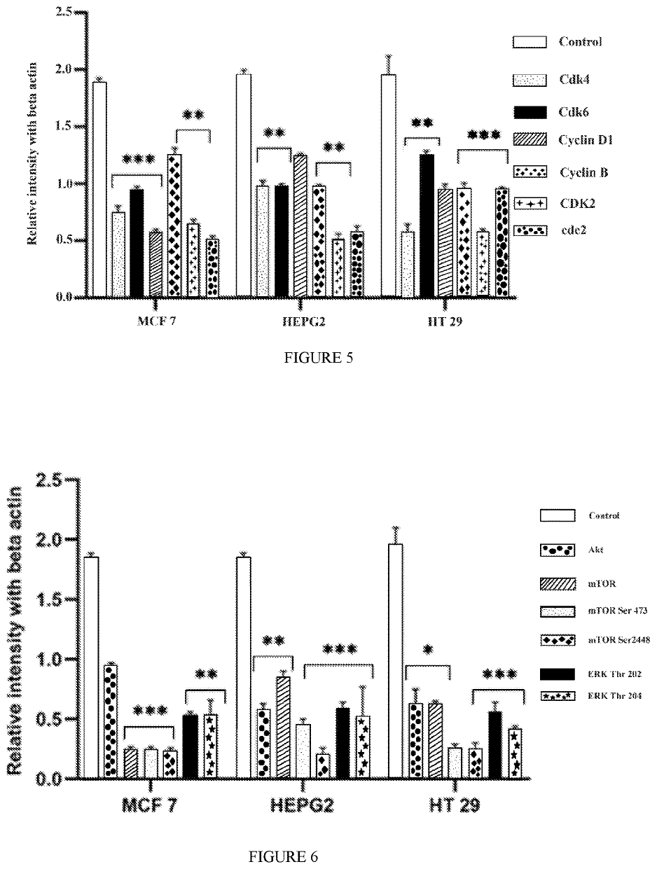

Flow cytometry analysis was performed to verify whether COMPOUND I induced apoptosis was related to cell cycle arrest. Cell cycle distribution in KRAS Mutated cell lines was quantified after treatment with different COMPOUND I concentrations. Flow cytometry analysis showed an increase in the percentage of G2/M arrest phase in cell line compared to control cell line. The number of cells in the G2/M phase increased (76.89%), in COMPOUND I treated cells in a dose dependent manner (FIG. 3). To further determine the effects of COMPOUND I on cell progression through G2/M-phase, its effects were investigated on protein levels of the p53, p21, p27 (FIG. 4), Cyclin B1, and cdc2 genes, all of which regulate transition through the G2 checkpoint As shown in FIG. 5, the expression levels of Cyclin B, and cdc2, CDK2 decreased significantly after treating with COMPOUND I in a dose-dependent fashion. Greater expression levels of p21, p27 and p53 were recorded in COMPOUND I treated KRAS Mutated cell lines G2 cells. Although an increase in cell population at G2/M phase was found, 23.98% of cells showed G1/S phase arrest (FIG. 3). Therefore, it was attempted to determine whether COMPOUND I treatment also can influence the expression level of G1/S phase cell cycle regulatory proteins. As shown in FIG. 4, the expression levels of CDK4, CDK6, Cyclin D1, Cyclin D3, pRb decreased significantly after treating with COMPOUND I in a dose-dependent manner. Control cell lines treated with COMPOUND I were better protected against changes in cell-cycle regulation and apoptosis compared with KRAS Mutated cell lines treated with COMPOUND I.

### Example 4

Effect of COMPOUND I on mTOR Pathway

In order to better understand the molecular basis of COMPOUND I induced cell cycle arrest and apoptosis, the expression of Akt and mTOR was investigated with various concentrations of COMPOUND I. Akt/mTOR is an important cell survival pathway and activated Akt inhibits apoptosis leading to cell survival. As shown in FIG. 6, activation of both AKT, as well as mTOR was suppressed by COMPOUND I in a concentration-dependent manner whereas total Akt and mTOR levels remained constant. It was found that COMPOUND I suppressed the phosphorylation of Akt and mTOR at Serine 473 and Serine 2448 residues respectively. The phosphorylation of ERK was also suppressed at both Threonine 202 and Tyrosine 204 upon COMPOUND I treatment. Additionally, it was examined whether COMPOUND I effect involved ERK signaling pathway and found the decreased activation of ERK. The results indicate that besides downregulating antiapoptotic gene products, COMPOUND I also mediates its effects through downregulation of Akt/mTOR cell signaling pathway

### Example 5

### Anti-Proliferative Effects Induced by the Selected Compounds

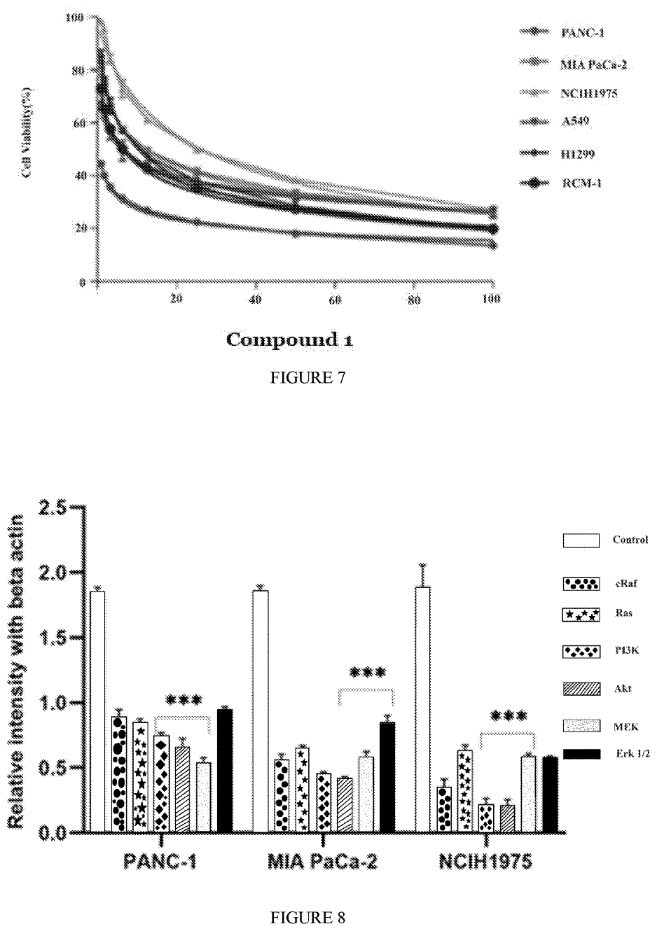

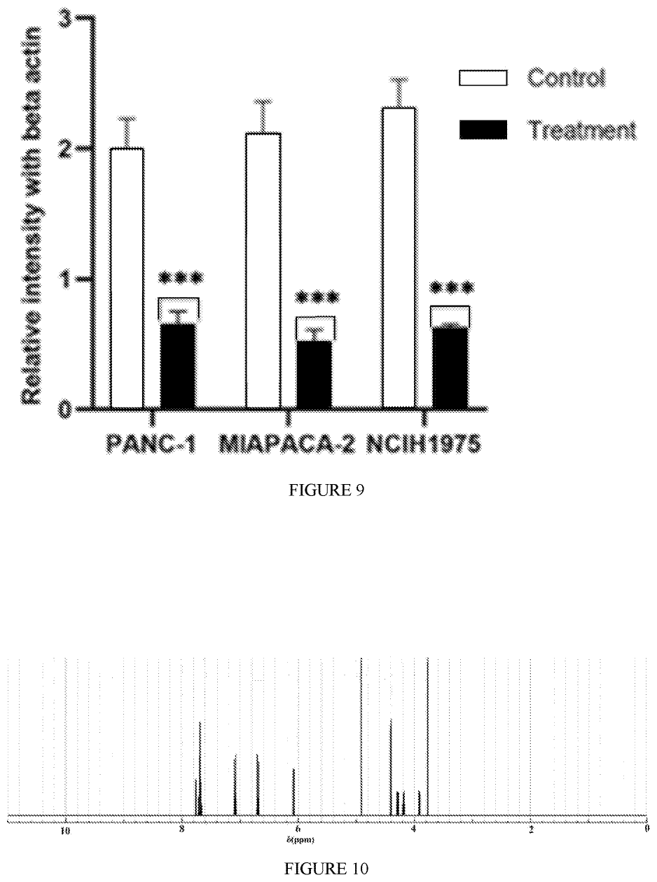

The inhibition of compounds COMPOUND I on the cellular proliferation of six cell lines were measured: The three cell lines in the signaling studies (PANC-1, MIA PaCa-2, NCIH1975), and the non-small cell lung cancer lines A549 (K-Ras G12S mutant) and NCI-H1299 (N-Ras Q61K mutant). FIG. 7 and IC50 values measured in these studies. COMPOUND I inhibit cellular proliferation in a dose dependent manner with similar IC50 values, ranging from approximately 0.45 μM to 1.20 μM in all tested cell lines. Consistent with the notion that these compounds are pan-Ras superfamily inhibitors, it is reasonable to speculate that they can inhibit pathways downstream of several members of the Rassuperfamily.

### Example 6

### Inhibition of Ras Downstream Pathways

The inhibitory effects of compounds COMPOUND I was tested on Ras-associated signal transduction pathways in three different human cell lines: the pancreatic cell-lines PANC-1 (K-Ras G12D mutant), MIA PaCa-2 (K-Ras G12C mutant), and the non-small cell lung cancer cell-line NCI-H1975 (KRas WT). FIG. 8 illustrate the inhibitory effects of COMPOUND I on phosphorylation and activation of MEK, Erk1/2 and Akt in those three cell lines. As shown in FIG. 2, compound COMPOUND I inhibited phosphorylation and activation of MEK, Erk1/2 and Akt in PANC-1, MIA PaCa-2 and NCI-H1975 cell lines in a dose-dependent manner with similar IC50s values (about 1-3 μM). These effects would be expected from a Ras inhibitor that is an upstream blocker of the two pathways and support the notion that COMPOUND I induces an inactive conformation of Ras through a consistent binding mode, regardless of different mutations. In contrast, the IC50s of compound COMPOUND I on the phosphorylation and activation of MEK and Erk1/2 were approximately 2 μM in PANC-1 and MIA PaCa-2 and 3 μM in NCI-H1975; and, of Akt, were approximately 5 μM in PANC-1 and MIA PaCa-2 and 5 μM in NCIH1975 (FIG. 8).

### Example 7

### Inhibition of the Ras-GTP Complex

As a follow up to the downstream assays, the ability of COMPOUND I was also measured to inhibit Ras-GTP complex formation in the three tested cell-lines (PANC-1, MIA PaCa-2 and NCIH1975) using a Ras pull-down assay. As shown in FIG. 4, compounds prevent Ras-GTP complex formation with IC50 values in the range of 2-3 μM. These values correlate well with the IC50 values obtained for the inhibition of MEK, Erk1/2 and Akt phosphorylation presented in FIGS. 2 and 3. The detected inhibition of Ras-GTP complex formation strongly suggests that both compounds bind directly to the GTP-binding site of Ras proteins and that, upon binding, the tested compounds induce a Ras inactive conformation, with a similar effect to the GDP-bound state.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope being indicated by the following claims.

We claim:

1. A compound of structural formula I: 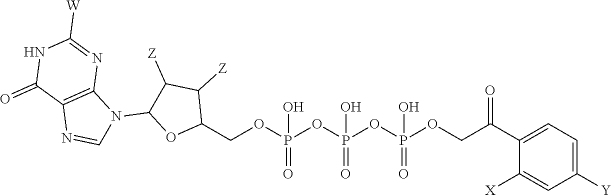
Formula I
or a pharmaceutically acceptable salt thereof; wherein
W is selected from a group comprising of substituted saturated/unsaturated branched/linear aliphatic amine, substituted aromatic amine, unsubstituted saturated/unsaturated branched/linear aliphatic amine, unsubstituted aromatic amine;
X is selected from a group comprising of H, substituted/unsubstituted saturated/unsaturated branched/linear aliphatic alkyl group with at least one carbon atom, substituted/unsubstituted saturated/unsaturated branched/linear aliphatic alkoxy group with at least one carbon atom, substituted/unsubstituted aryl group, substituted/unsubstituted aryloxy group;
Y is selected from a group comprising of H, substituted/unsubstituted saturated/unsaturated branched/linear aliphatic alkyl group bonded to a carbonyl group, substituted/unsubstituted aryl group bonded to a carbonyl group;
Z is selected from a group comprising of H, OH.

2. The compound as claimed in claim 1 wherein the said W is substituted aromatic amine or a pharmaceutically acceptable salt thereof.

3. The compound as claimed in claim 1 wherein the said W is N-bonded p-Hydroxyaniline.

4. The compound as claimed in claim 1 wherein the said X is unsubstituted saturated linear aliphatic alkoxy group with at least one carbon atom or a pharmaceutically acceptable salt thereof.

5. The compound as claimed in claim 1 wherein the said X is Methoxy group.

6. The compound as claimed in claim 1 wherein the said Y unsubstituted saturated linear aliphatic alkyl group bonded to a carbonyl group or a pharmaceutically acceptable salt thereof.

7. The compound as claimed in claim 1 wherein the said Y is Acetyl group.

8. The compound as claimed in claim 1 wherein the said Z is hydroxyl group or a pharmaceutically acceptable salt thereof.

9. The compound as claimed in claim 1 wherein the said Z is hydroxyl group.

10. The compound as claimed in claim 1 comprises of structure of compound I

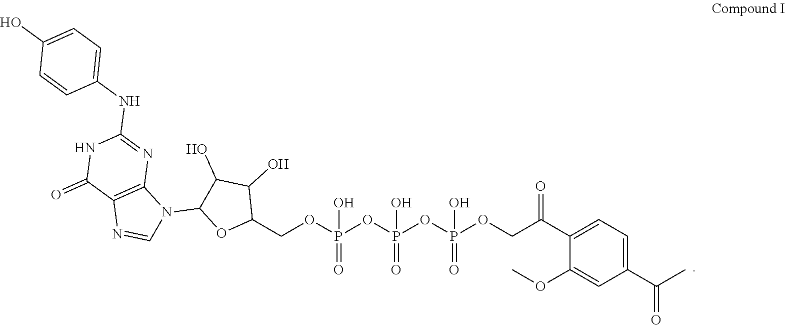 [SMILES: COc1cc(C(C)=O)ccc1C(=O)COP(=O)(O)OP(=O)(O)OP(=O)(O)OCC1OC(n2cnc3c(=O)[nH]c(Nc4ccc(O)cc4)nc32)C(O)C1O]

11. A pharmaceutical composition comprising a therapeutically effective amount of compound of claim 1, or a therapeutically effective amount of pharmaceutically acceptable salt thereof, and a pharmaceutically acceptable carrier.

12. A pharmaceutical composition comprising a therapeutically effective amount of compound of claim 10, and a pharmaceutically acceptable carrier.
